(19) United States
(12) Patent Application Publication
(10) Pub. No.: US 2021/0045697 A1
(43) Pub. Date: Feb. 18, 2021

(54) METHOD AND SYSTEM FOR PAIRING PHYSIOLOGICAL SIGNAL

(71) Applicant: Sil Radar Technology Inc., Kaohsiung City (TW)

(72) Inventors: Chang-Jen Wang, Kaohsiung City (TW); Chao-Kai Wen, Kaohsiung City (TW); Sheng-You Tian, Kaohsiung City (TW); Yi-Ting Tseng, Kaohsiung City (TW); Chan-Hung Lee, Kaohsiung City (TW)

(21) Appl. No.: 16/740,577

(22) Filed: Jan. 13, 2020

(30) Foreign Application Priority Data
Aug. 12, 2019 (TW) 108128454

(51) Int. Cl. A61B 5/00; A61B 5/0245

(52) U.S. Cl. CPC A61B 5/7285; A61B 5/0015; A61B 5/0013; A61B 5/0245; A61B 5/0006

(57) ABSTRACT

A method disclosed in the present invention utilizes an image captured by an image capture device to assist physiological signal paring. Multiple vital signs in the physiological signal detected by a radar are able to be paired with multiple subjects in the image one on one accurately to realize continuous tracking of vital signs of multiple subjects.

### FIELD OF THE INVENTION

This invention generally relates to method and system for pairing physiological signal, and more particularly to method and system for pairing physiological signal with the assistance of images.

### BACKGROUND OF THE INVENTION

Conventional skin-contact sensors for physiological signal detection, e.g. electrocardiography (ECG) and photoplethysmograpgy (PPG), have to directly adhere to human skin for accurate detection, and only one target subject can be detected at the same time so they are unavailable on wide range detection, such as abnormal heart rate detection in airport or hospital, and life detection at a disaster site. Different to contact type, non-contact sensors for physiological signal detection can detect multiple subjects located within signal range at the same time by using the Doppler Effect of wireless signals, however, they cannot distinguish vital signs of different subjects. For this reason, conventional non-contact sensors are usually applied to monitor sign of single subject, the monitoring of vital signs of multiple subjects by using non-contact sensor is still unavailable.

### SUMMARY

One objective of the present invention is to pair vital signs and subjects according to variations of images and vital signs over time and subjects position in the space by using time and space pairing modules of a compute device.

A method for pairing physiological signal of the present invention includes steps of: receiving an image and a physiological signal by using a compute device, an subject recognition module of the compute device is configured to identify a number and a position of one or a plurality of subjects in the image, and a vital sign acquisition module of the compute device is configured to extract one or a plurality of vital signs having a number the same as the number of the subject or the plurality of subjects from the physiological signal; determining whether the plurality of subjects are present in the image by using the subject recognition module, wherein when only the subject is recognized, a time pairing module of the compute device is configured to pair the vital sign in the physiology signal and the subject, and wherein when the plurality of subjects are recognized, a space pairing module of the compute device is configured to pair the plurality of vital signs and the plurality of subjects depending on an energy level of each of the plurality of vital signs and the position of each of the plurality of subjects; and determining whether there is one or a plurality of additional subjects according to the image at different times by using the subject recognition module, wherein when only the additional subject is recognized, the time pairing module is configured to pair an additional vital sign in the physiological signal and the additional subject, and wherein when the plurality of additional subjects are recognized, the space pairing module is configured to pair a plurality of additional vital signs and the plurality of additional subjects depending on the energy level of each of the plurality of additional vital signs and the position of each of the plurality of additional subjects.

A system for pairing physiological signal of the present invention includes an image capture device configured to capture an image, a radar configured to detect a physiological signal, and a compute device including a subject recognition module, a vital sign acquisition module, a time pairing module and a space pairing module. The subject recognition module is electrically connected to the image capture device for receiving the image, and the subject recognition module is configured to identify a number and a position of one or a plurality of subjects in the image. The vital sign acquisition module is electrically connected to the radar and the subject recognition module for receiving the physiological signal, and the vital sign acquisition module is configured to extract one or a plurality of vital signs having a number the same as the number of the subject or the plurality of subjects from the physiological signal. The time pairing module is electrically connected to the subject recognition module and the vital sign acquisition module for receiving the position of the subject and the vital sign, and the time pairing module is configured to pair the vital sign in the physiology signal and the subject. The space pairing module is electrically connected to the subject recognition module and the vital sign acquisition module for receiving the position of each of the plurality of subjects and receiving the plurality of vital signs from the vital sign acquisition module, and the space pairing module is configured to pair the plurality of vital signs and the plurality of subjects depending on an energy level of each of the plurality of vital signs and the position of each of the plurality of subjects.

Multiple vital signs and subjects are able to be paired one on one accurately owing to the present invention utilizes the image captured by the image capture device to assist the pairing of the physiological signal detected by the radar. The method and system disclosed in the present invention are helpful for non-contact detection of physiological signals of multiple subjects.

### DESCRIPTION OF THE DRAWINGS

FIG. 1 is a flowchart illustrating a method for pairing physiological signal in accordance with one embodiment of the present invention.

FIG. 2 is a block diagram illustrating a system for pairing physiological signal in accordance with one embodiment of the present invention.

FIG. 3 is a schematic diagram illustrating one type of combined image in accordance with one embodiment of the present invention.

FIG. 4 is a schematic diagram illustrating one type of combined image in accordance with one embodiment of the present invention.

FIG. 5 is a schematic diagram illustrating detection ranges of a radar having high directivity antenna or beamforming antenna array in accordance with one embodiment of the present invention.

FIG. 6 is a practical combined image obtained by one embodiment of the present invention.

### DETAILED DESCRIPTION OF THE INVENTION

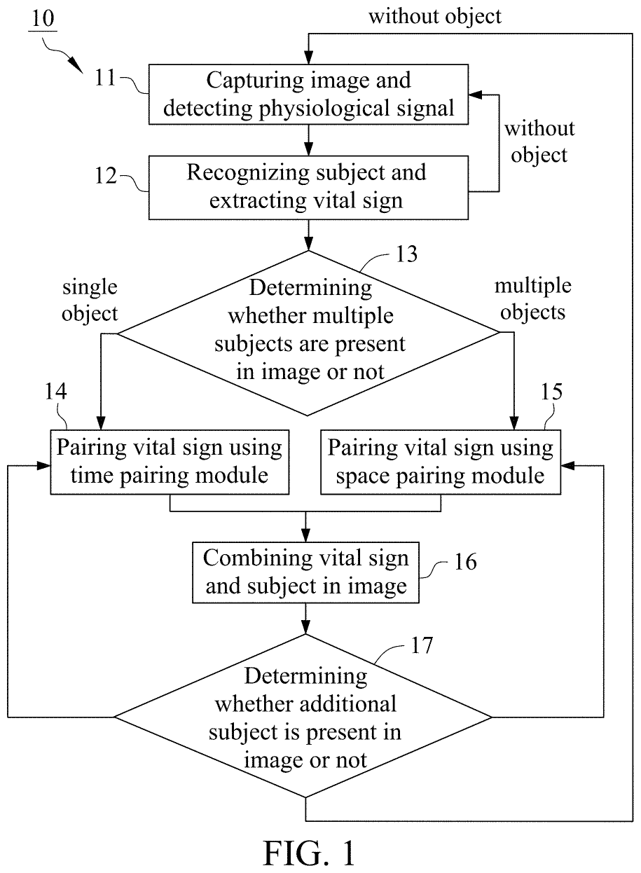

FIG. 1 is a flowchart of a method 10 for pairing physiological signal in accordance with one embodiment of the present invention. The method 10 includes a step 11 of capturing image and detecting physiological signal, a step 12 of recognizing subject and extracting vital sign, a step 13 of determining whether multiple subjects are present in image, a step 14 of pairing vital sign using time pairing module, a step 15 of pairing vital sign using space pairing module, a step 16 of combining vital sign and subject in image and a step 17 of determining whether additional subject is present in image.

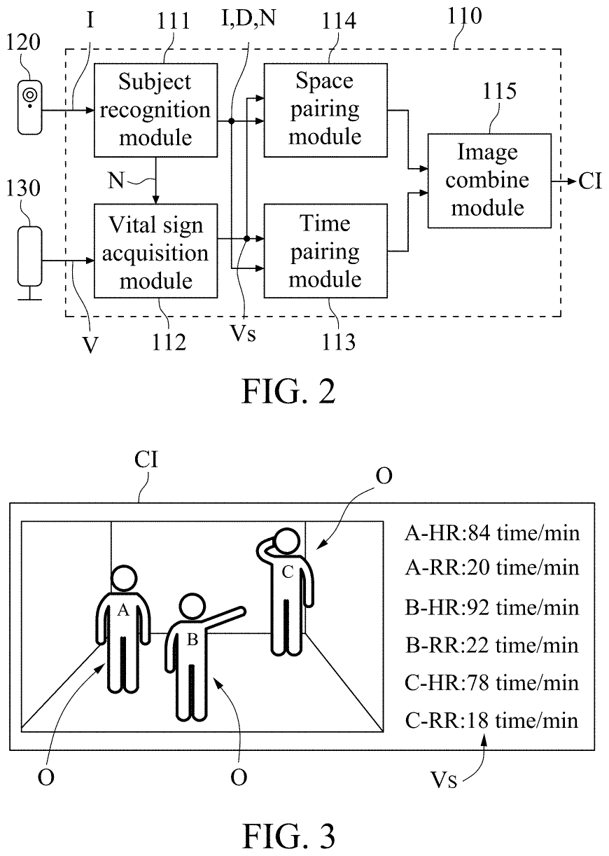

With reference to FIGS. 1 and 2, an image capture device 120 is configured to capture an image I and a radar 130 is configured to detect a physiological signal V in the step 11. The image capture device 120 may be a depth camera, a thermal camera or an optical camera, and preferably, the image I captured by the image capture device 120 is a dynamic image that changes over time. The radar 130, e.g. Doppler radar, self-injection-locked radar or ultra-wideband radar, is utilized to detect physiological signal(s) of subject(s) around it by the Doppler Effect of radio waves. In this embodiment, the image capture device 120 is a thermal camera able to capture a dynamic thermographic image for confirming number and monitoring body temperature of subject(s) according to temperature distribution, and the radar 130 is a self-injection-locked radar which is highly sensitive to vital sign and beneficial to detect vital signs of multiple subjects from the physiological signal V.

With reference to FIGS. 1 and 2, a subject recognition module 111 of a compute device 110 is configured to receive the image I from the image capture device 120 and a vital sign acquisition module 112 of the compute device 110 is configured to receive the physiological signal V from the radar 130 in the step 12. The compute device 110 may be single chip having logic circuits, programmable single chip or computer, and the modules of the compute device 110 may be arithmetic logic unit, program or software. A number N and a position P of the subject(s) O in the image I are identified by the subject recognition module 111. If the image I is a thermal image captured by a thermal camera, the subject recognition module 111 utilizes temperature distribution of the image I to determine the number N of the subject(s) O and utilizes distance measurement of the subject(s) O by using a laser detector (not shown) to estimate the position P of the subject(s) O in the image I. If the image capture device 120 is a depth camera or optical camera, the number N of the subject(s) O in the image I can be identified by human skeleton or facial recognition, and the distance of the subject(s) O can be measured according to view angles of multiple images captured by multiple lenses of camera to estimate the position P of the subject(s) O.

The vital sign acquisition module 112 is configured to extract one or more vital signs Vs having a number the same as the number N of the subject(s) from the physiological signal V. In this embodiment, the vital sign acquisition module 112 analyzes frequency components of the physiological signal V by a Fast Fourier Transform (FFT) to acquire the vital sign Vs of each of the subjects O, the vital sign Vs is respiration or heartbeat. However, it is difficult to directly know how many vital signs are involved in the physiological signal V by frequency spectral analysis due to the vibration frequencies caused by respiration or heartbeat of different subjects are similar. Consequently, the vital sign acquisition module 112 of this embodiment extracts the vital sign(s) Vs having a number the same as the number N of the subject(s) O determined by the subject recognition module 111. Further, if the subject recognition module 111 doesn't recognize any subject in the step 12, the step 11 will restart to capture the image I by the image capture device 120 and detect the physiological signal V by the radar 130 continuously.

With reference to FIGS. 1 and 2, in the step 13, the subject recognition module 111 is configured to determine whether multiple subjects O are present in the image I. Next, the step 14 or 15 is processed after the step 13. When only one subject O is recognized in the image I, a time pairing module 113 of the compute device 110 is configured to pair vital sign in the step 14. On the other hand, a space pairing module 114 of the compute device 110 is configured to pair vital signs in the step 15 when there are multiple subjects O recognized in the image I.

With reference to FIGS. 1 and 2, in the step 14, the time pairing module 113 receives the image I and the information that there is only one subject O in the image I from the subject recognition module 111, and a single vital sign Vs extracted from the physiological signal V by the vital sign acquisition module 112 is transmitted to the time pairing module 113 to pair with the single subject O.

If multiple subjects O are present in the image I, multiple vital signs Vs having a number the same as the number N of the subjects O are extracted from the physiological signal V by the vital sign acquisition module 112, but they cannot be paired to the subjects O directly. For this reason, the space pairing module 114 is provided to pair the vital signs Vs and the subjects O according to an energy level of each of the vital signs Vs and the position P of each of the subjects O in the step 15. The longer the distance between the subject O and the radar 130, the smaller energy level of the vital sign Vs due to the radar 130 detects the vital signs Vs by transmitting wireless signals to the subjects O and receiving reflected signals from the subjects O in this embodiment. Consequently, the space pairing module 114 is configured to pair the vital signs Vs in order of the energy level from high to low with the subjects O in order of the position P from near to far one on one.

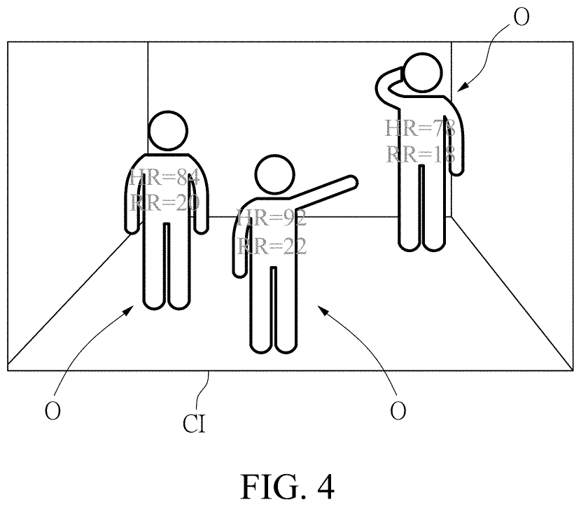

With reference to FIGS. 1 and 2, the step 16 is proceeded after the step 14 or 15. An image combine module 115 of the compute device 110 is configured to combine the vital sign(s) Vs and the subject(s) O in the image I paired by the time pairing module 113 or the space pairing module 114 and output a combined image CI. There are different types of combined images as shown in FIGS. 3 and 4. FIG. 3 is one possible combined image CI that the subjects O are numbered and the vital signs Vs paired with the subjects O are displayed on right side, and FIG. 4 is another possible combined image CI that the vital signs Vs are displayed on the subjects O directly. The type of the combined image CI is depending on the monitor size and user requirement, not restricted in the present invention.

With reference to FIGS. 1 and 2, the image capture device 120 and the radar 130 work continuously such that the subject recognition module 111 can determine whether there is one or more additional subjects O according to the images captured at different times in the step 17. If the subject recognition module 111 determines there is only one additional subject O, no additional subject O or less subjects O in the image I, the step 14 of pairing vital sign using the time pairing module 113 is proceeded after the step 17. In the other hand, the step 15 of pairing vital signs using the space pairing module 114 is proceeded after the step 17 when there are multiple additional subjects O recognized in the image I. Otherwise, if there is no subject recognized in the image I, the step 11 is proceeded after the step 17 to re-pair the vital sign(s) Vs and the subject(s) O recognized in the image I.

With reference to FIG. 1, if one or more additional subjects O are recognized in the image I in the step 17, the vital sign acquisition module 112 needs to extract one or more additional vital signs Vs from the physiological signal V. More than one vital signs Vs, including the original vital sign and the additional vital sign(s), are Obtained, and it is necessary to distinguish the additional vital sign(s) from the original vital sign before the step 14 or 15. In this embodiment, the vital sign acquisition module 112 is configured to identify a difference between the vital signs Vs before and after appearance of the additional subject (s) O and distinguish which one is the additional vital sign Vs by the difference. The vital sign acquisition module 112 determines the vital sign Vs having the smallest difference is the original vital sign Vs because the vital sign does not change much in a short period of time, and determines the other vital sign(s) Vs is the additional vital sign(s) Vs. For instance, the vital sign acquisition module 112 extracts a heart rate of 83 beats per minute before appearance of the additional subjects O and extracts three heart rates of 84, 88 and 78 beats per minute after appearance of the additional subjects O, and determines the heart rate of 84 beats per minute, having the smallest difference (1 beat per minute), is the original vital sign Vs and the heart rates of 88 and 78 beats per minute, having the difference of 5 beats per minute, are the additional vital signs Vs.

With reference to FIGS. 1 and 2, the vital sign acquisition module 112 extracts one additional vital sign Vs from the physiological signal V when a single additional subject O is recognized, and in the step 14, the additional vital sign Vs is paired with the additional subject O and the original vital sign Vs is paired with the original subject O by the time pairing module 113. While the number of the subjects O is decreased or not changed, there is no additional vital sign Vs extracted from the physiological signal V by the vital sign acquisition module 112 and the original vital sign(s) Vs is paired with the original subject(s) O by the time pairing module 113.

With reference to FIGS. 1 and 2, if multiple additional subjects O are recognized, the vital sign acquisition module 112 extracts multiple additional vital signs Vs from the physiological signal V, and the space pairing module 114 is configured to pair the additional vital signs Vs to the additional subjects O one on one based on energy level of each of the additional vital signs Vs and distance of each of the additional subjects O in the step 15. The space pairing module 114 is configured to pair the additional vital signs Vs in order of the energy level from high to low and the additional subjects O in order of the position P from near to far, and configured to pair the original vital sign(s) Vs with the original subject(s) O. After pairing, the image combine module 115 combines the vital signs and the subjects in the image I to output the combined image CI.

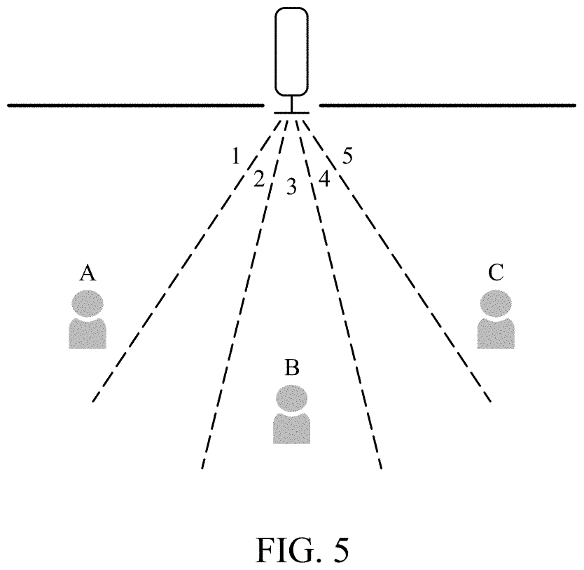

With reference to FIG. 5, in different embodiment, the radar 130 configured to detect the physiological signal V is a radar having high directivity antenna or antenna array, and wireless signal from the radar 130 has a beam width not wide enough to cover the whole capture region of the image capture device 120. As a result, directing the radar 130 to different positions by rotating antenna or changing beam direction of antenna array is required to detect all subjects within the capture region. If the image capture device 120 captures an image involved the subjects A, B and C but the beam width of wireless signal from the radar 130 only can cover one of the sub-regions 1, 3, 4 or 5, the capture region of the image capture device 120 can be divided into 5 sub-regions in advance. When there is only one subject recognized in the sub-region by the subject recognition module 111, the radar 130 is directed to the sub-region, where the subject is located within, to detect the physiological signal V, the vital sign acquisition module 112 extracts a single vital sign Vs from the physiological signal V, and the time pairing module 113 pairs the vital sign Vs acquired from the sub-region and the subject. As shown in FIG. 5, when there is only one subject. B recognized in the sub-region 3 by the subject recognition module 111, the radar 130 is directed toward the sub-region 3 to detect the physiology signal V, then the vital sign acquisition module 112 extracts a single vital sign Vs from the physiological signal V and the time pairing module 113 pairs the single vital sign Vs and the single subject B. Reversely, if the subject recognition module 111 recognizes more than one subjects B in the sub-region 3, the space pairing module 114 is provided to pair the vital signs Vs and the subjects B depending on the energy level of each of the vital signs Vs.

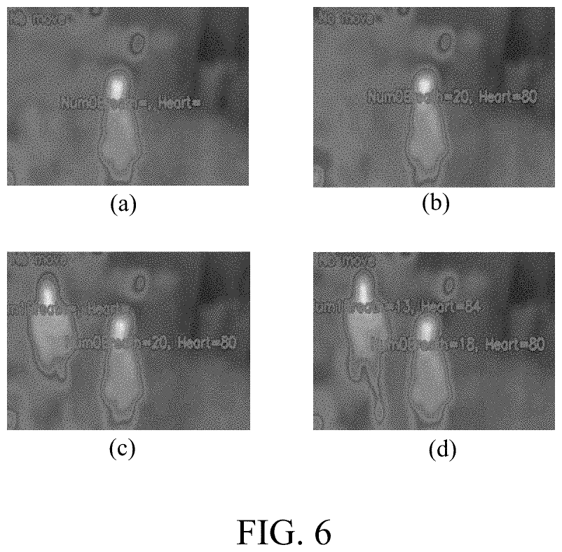

FIG. 6 is a combined image CI of a thermal image captured by a thermal camera and vital signs detected by a self-injection-locked radar. (a) shows a person enters the thermal camera range and the pairing is not completed yet so there is no vital sign displayed in the image, and then the vital sign is displayed on the person instantaneously as shown in (b) when the pairing is completed. (c) and (d) show there is another person entering the thermal camera range before and after the pairing of additional vital sign. As shown in (d), the pairing of multiple vital signs and multiple subjects are completed and the vital signs are displayed on the paired person, respectively.

The present invention utilizes the image I captured by the image capture device 120 to assist the pairing of the subject(s) O and the vital sign(s) Vs in the physiological signal V detected by the radar 130 so that the pairing of multiple vital signs and subjects is available for non-contact detection of physiological signals.

While this invention has been particularly illustrated and described in detail with respect to the preferred embodiments thereof, it will be clearly understood by those skilled in the art that is not limited to the specific features shown and described and various modified and changed in form and details may be made without departing from the spirit and scope of this invention.

What is claimed is:

1. A method for pairing physiological signal, the method comprising steps of:
receiving an image and a physiological signal by using a compute device, an subject recognition module of the compute device is configured to identify a number and a position of one or a plurality of subjects in the image, and a vital sign acquisition module of the compute device is configured to extract one or a plurality of vital signs having a number the same as the number of the subject or the plurality of subjects from the physiological signal;
determining whether the plurality of subjects are present in the image by using the subject recognition module, wherein when only the subject is recognized, a time pairing module of the compute device is configured to pair the vital sign in the physiology signal and the subject, and wherein when the plurality of subjects are recognized, a space pairing module of the compute device is configured to pair the plurality of vital signs and the plurality of subjects depending on an energy level of each of the plurality of vital signs and the position of each of the plurality of subjects; and
determining whether there is one or a plurality of additional subjects according to the image at different times by using the subject recognition module, wherein when only the additional subject is recognized, the time pairing module is configured to pair an additional vital sign in the physiological signal and the additional subject, and wherein when the plurality of additional subjects are recognized, the space pairing module is configured to pair a plurality of additional vital signs and the plurality of additional subjects depending on the energy level of each of the plurality of additional vital signs and the position of each of the plurality of additional subjects.

2. The method in accordance with claim 1, wherein when there is the at least one additional subject recognized in the image, the vital sign acquisition module is configured to identify a difference between the vital signs before and after the appearance of the at least one additional subject and configured to distinguish an additional vital sign from the vital signs in the physiological signal according to the difference.

3. The method in accordance with claim 2, wherein the vital sign acquisition module is configured to determine the vital sign having the smallest difference as the original vital sign and determine the other vital sign as the additional vital sign.

4. The method in accordance with claim 1, wherein the image is captured by an image capture device which is one of a depth camera, a thermal camera, and an optical camera.

5. The method in accordance with claim 1, wherein the physiological signal is detected by a radar which is one of a Doppler radar, a self-injection-locked radar, and an ultra-wideband radar.

6. The method in accordance with claim 1, wherein the space pairing module is configured to pair the vital signs and the subjects according to an angle of each of the vital signs and a region where each of the subjects is located in the image.

7. The method in accordance with claim 6, wherein the physiological signal is detected by a radar having a high directivity antenna or a beamforming antenna array.

8. The method in accordance with claim 1 further comprising a step of combining the paired vital sign(s) to the subject(s) in the image by using an image combine module of the compute device, wherein the image combine module is configured to output a combined image.

9. A system for pairing physiological signal, comprising:
an image capture device configured to capture an image;
a radar configured to detect a physiological signal; and
a compute device including an subject recognition module, a vital sign acquisition module, a time pairing module and a space pairing module;
wherein the subject recognition module is electrically connected to the image capture device for receiving the image, and the subject recognition module is configured to identify a number and a position of one or a plurality of subjects in the image;
wherein the vital sign acquisition module is electrically connected to the radar and the subject recognition module for receiving the physiological signal, and the vital sign acquisition module is configured to extract one or a plurality of vital signs having a number the same as the number of the subject or the plurality of subjects from the physiological signal;
wherein the time pairing module is electrically connected to the subject recognition module and the vital sign acquisition module for receiving the position of the subject and the vital sign, and the time pairing module is configured to pair the vital sign in the physiology signal and the subject;
wherein the space pairing module is electrically connected to the subject recognition module and the vital sign acquisition module for receiving the position of each of the plurality of subjects and receiving the plurality of vital signs from the vital sign acquisition module, and the space pairing module is configured to pair the plurality of vital signs and the plurality of subjects depending on an energy level of each of the plurality of vital signs and the position of each of the plurality of subjects.

10. The system in accordance with claim 9, wherein the compute device further includes an image combine module which is electrically connected to the time pairing module and the space pairing module and configured to combine the paired vital sign(s) to the subject(s) in the image to output a combined image.
